(19) United States
(12) Patent Application Publication
(10) Pub. No.: US 2004/0006053 A1
(43) Pub. Date: Jan. 8, 2004

(54) Method of long-term treatment of Graft-Versus-Host Disease using topical active corticosteriods

(75) Inventor: George B. McDonald, Bellevue, WA (US)

Correspondence Address: KATTEN MUCHIN ZAVIS ROSENMAN, Attention: Patent Administrator, Suite 1600, 525 West Monroe Street, Chicago, IL, 60661-3693, US

(73) Assignee: Enteron Pharmaceuticals, Inc.

(21) Appl. No.: 10/613,788

(22) Filed: Jul. 3, 2003

Related U.S. Application Data
(63) Continuation of application No. 09/753,814, filed on Jan. 3, 2001, now abandoned.
(60) Provisional application No. 60/233,194, filed on Sep. 15, 2000.

(51) Int. Cl. A61K 31/573

(52) U.S. Cl. 514/179; 514/170

(57) ABSTRACT

A method for long-term therapy using corticosteriods to treat tissue damage associated with graft-versus-host disease in a patient having undergone hematopoietic cell transplantation, and host-versus-graft disease in a patient having undergone organ allograft transplantation. The method includes orally administering to the patient a therapeutically effective amount of a topically active corticosteroid, such as beclomethasone dipropionate, from the 29^{th} day until the 56^{th} day following hematopoietic cell or organ allograft transplantation. Representative tissues includes tissue of the intestine and liver, while representative tissue damage includes inflammation thereof.

### FIELD OF THE INVENTION

This invention relates to the long-term treatment of Graft-Versus-Host Disease (GVHD) and more, particularly to the treatment of intestinal or gastrointestinal Graft-Versus-Host Disease by an orally effective therapeutic agent.

### BACKGROUND OF THE INVENTION

Graft-versus-host disease (GVHD) is a complication of allogeneic hematopoietic cell transplantation in which tissues of the host, most frequently the skin, liver and intestine, are damaged by lymphocytes from the donor. The risk and severity of this immune-mediated condition are directly related to the degree of mismatch between a host and the donor of hematopoietic cells. For example, GVHD develops in up to 30% of recipients of human leukocyte antigen (HLA)-matched sibling marrow, in up to 60% of recipients of HLA-matched unrelated donor marrow, and in a higher percentage of recipient of HLA-mismatched marrow. Patients with mild intestinal GVHD present symptoms of anorexia, nausea, vomiting, abdominal pain and diarrhea, whereas patients with severe GVHD are disabled by these symptoms. If untreated, symptoms of intestinal GVHD persist and often progress; spontaneous remissions are unusual. In its most severe form, GVHD leads to necrosis and exfoliation of most of the epithelial cells of the intestinal mucosa, a frequently fatal condition.

Standard initial treatment for acute GVHD includes systemic immunosuppressive agents, usually high-dose prednisone at 2 mg per kg per day added to prophylactic medications such as methotrexate, cyclosporine and tacrolimus. Prednisone achieves a complete and sustained remission of gastrointestinal symptoms in 50-70% of patients with GVHD. Patients who fail to respond receive therapy with additional immunosuppressive regimens, such as higher-dose prednisone, anti-thymocyte globulin, and investigational anti-T-cell monoclonal antibodies or immunotoxins. Unfortunately, the risks of prolonged immunosuppressive therapy are significant, especially among patients with immature marrow grafts. These risks include local and disseminated infection, the development of lymphoproliferative disease, and systemic glucocorticoid side effects such as hypothalamic-pituitary-adrenal axis suppression, myopathy, neuropsychiatric disease, and bone demineralization.

Recently, investigators have reported the results of a phase I trial of topically active corticosteroid, beclomethasone dipropionate (BDP), for the treatment of patients with intestinal GVHD (Baehr et al., Transplantation 60:1231-1238, 1995). In this trial, BDP capsules were given orally, 8 mg daily, half as enteric-coated capsules designed to dissolve in the alkaline pH of the upper small intestine, and half of the capsules that dissolve in the stomach. Significant improvement was found in the appetite, oral intake, nausea, and diarrhea over the course of therapy with oral BDP alone and with oral BDP added to prednisone therapy. However, the time to improvement in patients receiving BDP as monotherapy was 7-10 days, which is longer than the response usually seen with prednisone therapy.

A drawback with the above regimen is that treatment is initiated with BDP only after presentation of symptoms of intestinal GVHD, with typical patient enrollment at a mean of 58 days post-transplant (i.e., ranging from day 21-231 after transplant). The difficulty with treatment after presentation of intestinal GVHD symptoms is that significant inflammation and/or damage to the intestine has already occurred prior to initiation of therapy. Severe damage to the lining of the intestine is often fatal, as malnutrition, protein loss, and blood stream infections preclude regeneration of lining cells. This study did, however, provide evidence that oral BDP therapy was safe and effective in the treatment of mild-to-moderate intestinal GVHD, taken alone or when added to prednisone.

A related condition to GVHD is host-versus-graft disease (HVGD), also referred to as organ allograft rejection. HVGD disease may occur, for example, when a donor intestine is transplanted into a patient with a diseased intestine. In this case, cells of the patient's immune system (the host) may attack the foreign intestinal tissue (the graft). While intestinal transplantation is not routine at the present time, such techniques will likely become more common. Thus, prophylactic medications are needed to prevent HVGD for many of the reasons noted above with regard to GVHD.

While significant advances have been made with regard to the treatment of GVHD following bone marrow transplantation, there is still a need in the art for improved methods, particularly in the context of preventing the intestinal mucosal damage associated with the onset of GVHD. Such preventative methods should begin immediately following hematopoietic cell transplantation, and reduce tissue damage associated with the subsequent onset of GVHD. There is also a need for methods to prevent HVGD in the context of, for example, intestinal or liver transplantation. The present invention fulfills these needs and provides further related advantages.

### SUMMARY OF THE INVENTION

In brief, this invention discloses a method for long term treatment of tissue damage, particularly of the intestinal and/or liver, caused by graft-versus-host disease (GVHD) that commonly follows hematopoietic cell transplantation, or caused by host-versus-graft disease (HVGD) or organ allograft rejection.

Hematopoietic cell transplantation is the generic term that encompasses bone marrow transplantation, peripheral blood stem cell transplantation, umbilical vein blood transplantation, or any other source of pleuripotent hematopoietic stem cells. The method includes the oral administration of an effective amount of a topically active corticosteroid (abbreviated herein as “TAC”) to a patient having undergone hematopoietic cell transplantation. A representative TAC of this invention is beclomethasone dipropionate (BDP). Such long term administration starts following the hematopoietic cell transplantation and continues up to day 56 following the hematopoietic cell transplantation, thereby treating, delaying and/or reducing severity of the symptoms-normally associated with tissue damage caused by GVHD.

In one embodiment, the tissue damage is caused by intestinal inflammation associated with intestinal graft-versus-host disease in a patient having undergone hematopoietic cell transplantation. In this embodiment, an effective amount of a TAC is orally administered to a patient in need thereof from day 29 to day 56 following hematopoietic cell transplantation.

In another embodiment, the tissue damage is caused by HVGD or organ allograft rejection, including (but not limited to) intestinal or liver transplantation. In this embodiment, a an effective amount of a TAC is orally administered to a patient in need thereof from day 29 to day 56 following hematopoietic cell transplantation.

In more specific embodiments, the TAC is administered orally at a dosage of 4 mg/day to 12 mg/day in a form suitable for oral administration, such as capsules, pills, coated microspheres with specific dissolution qualities, or emulsions. Other agents may optionally also be included in such oral formulations.

These and other aspects of this invention will be evident upon reference to the following detailed description.

### DETAILED DESCRIPTION OF THE INVENTION

As mentioned above, this invention is directed to a method for long term treatment of tissue damage caused by graft-versus-host disease (GVHD) which commonly follows hematopoietic cell transplantation, as well as by host-versus-graft disease (HVGD) or allograft rejection which commonly follows organ transplantation. As used herein, the term “long-term treatment” means administration of an effective therapy to reduce the severity of the symptoms associated with GVHD after day 29 to day 56 following hematopoietic cell transplantation or HVGD following organ allograft transplantation.

In the context of this invention, “tissue” means intestinal mucosa or the small bile ducts in the liver. Intestinal mucosa includes mucosa of the esophagus, stomach, small intestine and colon. “Damage” to such tissue may range from mild inflammation to destruction of the mucosa of the intestine to fatal exfoliation of intestinal epithelial cells. Inflammation typically presents as fever, abdominal pain, nausea, vomiting, diarrhea, intestinal bleeding, and jaundice.

The method of the present invention employs oral administration of a long term treatment effective amount of a topically active corticosteroid (TAC) to a patient having undergone hematopoietic cell or organ allograft transplantation. Representative TACs include, but are not limited to, beclomethasone dipropionate, alclometasone dipropionate, busedonide, 22S busesonide, 22R budesonide, beclomethasone-17-monopropionate, clobetasol propionate, diflorasone diacetate, flunisolide, flurandrenolide, fluticasone propionate, halobetasol propionate, halcinocide, mometasone furoate, and triamcinalone acetonide. Such TACs are well known to those skilled in the field of, for example, intestinal disorders, and are commercially available from any number of sources. Suitable TACs of this invention have rapid first-pass metabolism in the intestine and liver, low systemic bioavailability, high topical activity, and rapid excretion (see, e.g., Thiesen et al., Alimentary Pharmacology & Therapeutics 10:487496, 1996) (incorporated herein by reference).

In one embodiment of this invention, the TAC is beclomethasone dipropionate (BDP). BDP is a compound which is available from a number of commercial sources, such as Schering-Plough Corporation (Kenilworth, N.J.) in bulk crystalline form, and has the following structure (i.e., beclomethasone 17,21-dipropionate):

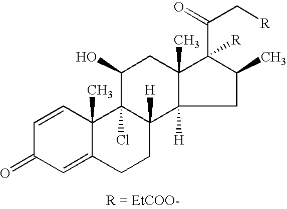

Patients having undergone hematopoietic cell or organ allograft transplantation, and which may thus be administered a TAC according to this invention, are allogenic hematopoietic cell recipients who have typically received marrow-ablative chemotherapy and/or total body irradiation followed by donor hematopoietic cell infusion, or patients having undergone intestinal or liver transplantation. Such procedures have been widely disclosed, and are well known to those skilled in this field.

Such patients receive a therapeutically acceptable amount of a TAC by oral administration. The TAC may be formulated for oral administration by techniques well known in the formulation field, including formulation as a capsule, pill, coated microsphere with specific dissolution qualities, or emulsion. Suitable capsules or pills generally contain from 1 mg to 2 mg TAC, and typically about 1 mg TAC, plus optional fillers, such as lactose, and may be coated with a variety of materials, such as cellulose acetate phthalate. By appropriate coating, such capsules, microspheres or pills may be made to dissolve within various location of the intestinal tract. For example, enteric-coated capsules prepared with a coating of cellulose acetate phthalate are known to dissolve in the alkaline environment of the small bowel, thus delivering its content to the small bowl and colon. Emulsions containing a TAC may also be employed for oral delivery, including optional emulsifying agents.

In addition to the TAC, acceptable carriers and/or diluents may be employed and are familiar to those skilled in the art. Formulations in the form of pills, capsules, microspheres, granules or tablets may contain, in addition to one or more TACs, diluents, dispersing and surface active agents, binders and lubricants. One skilled in the art may further formulate the TAC in an appropriate manner, and in accordance with accepted practices, such as those disclosed in Remington's Pharmaceutical Sciences, Gennaro, Ed., Mack Publishing Co., Easton, Pa., 1990 (incorporated herein by reference).

In the practice of this invention, a “long term therapeutically effective amount” of a TAC is administered to a patient in need thereof. In general terms, a long term therapeutically effective amount of a TAC is an amount which, when delivered orally, after day 29 to day 56 following hematopoietic cell transplantation, or associated with HVGD following organ allograft transplantation. Such an amount may be readily determined by one skilled in the art by well known dose-response investigations, and will generally range from 4 mg/day to 12 mg/day, and more typically range from 6 mg/day to 8 mg/day.

As optional components, other active long-term agents may be administered in combination with the TAC, including (but not limited to) prednisone, prednisolone, cyclosporine, methotrexate, tacrolimus and biological agents that affect T-lymphocytes.

In the context of GVHD, long term therapeutic administration of a TAC begins after the 29^{th} day after infusion of hematopoietic cells, and continues for 56 days after infusion of hematopoietic cells.

An important aspect of this invention is that the TAC is orally administered such that it is topically administered to the intestinal and/or liver tissue. Thus, oral administration, as that term is used herein, is not intended to encompass systemic administration, such as by intravenous injection. Rather, the TAC has little (if any) systemic availability, but high topical activity on intestinal and/or liver tissue. Such limited distribution results in fewer side effects, which is a significant advantage of this invention.

In addition to differences with regard to location and timing of administration, there is also a biological basis between short-term and long term treatment regiments. Corticosteriods have been used in the acute or short term management of GVHD. Conversely, long term therapy using corticosteriods is not appropriate use to the systemic side effects of compounds, such as prednisone. Therefore, using BDP for long term therapy, it is able to control the symptoms of GVHD without having systemic exposure to steriod toxicity.

In treatment, the objectives are to suppress a wide variety of biological events that have already resulted in tissue destruction, for example, the generation of inflammatory cytokines, the recruitment of additional inflammatory cells to the site of injury, the destruction of the barrier function of the intestinal mucosa (the lining), the passage of bacteria and toxins through the damaged intestinal mucosa, the up-regulation of biologic responses to bacteria and endotoxin, and the widespread organ responses to these events (such as leaky blood vessels, increased cardiac output, decreased systemic vascular resistance, diffuse lung injury, and renal insufficiency). When a patient has GVHD, treatment is successful only 50-75% of the time; the remainder of the patients generally die.

By appropriate formulation of the TAC (such as enterically coated capsules), it can be delivered to all of the mucosal surface of the intestine in high doses. Thus, the TAC can achieve high concentrations in the intestinal mucosa where this initiating alloimmune recognition event is taking place. It is believed that blunting the initiating event prevents the large cascade of biologic events that make up the syndromes of GVHD and HVGD.

It will be appreciated that, although specific embodiments of this invention have been described herein for purpose of illustration, various modifications may be made without departing from the spirit and scope of the invention.

We claim:

1. A method of treating a patient requiring long-term therapy following hematopoietic cell transplantation having graft-versus-host disease (GVHD) or folowing organ allograft transplantation having host-yersus-graft disease (HVGD)c the method comprising long term topical oral administration of a topically active corticosteroid.

2. The method of claim 1 wherein the topically active corticosteroid is administered orally at a dosage of 4 mg per day to 12 mg per day.

3. The method of claim 1 wherein the patient has tissue damage and the tissue is intestinal mucosa.

4. The method of claim 1 wherein the patient has tissue damage and the tissue is small bile ducts in the liver.

5. The method of claim 1 wherein the patient has tissue damage and the tissue damage is inflammation.

6. The method of claim 1 wherein the patient has tissue damage and the tissue damage is destruction of the mucosa of the intestine.

7. The method of claim 1 wherein the topically active corticosteriod is administered orally from day 29 to day 56 following hematopoietic cell transplantation.

8. The method of claim 1 wherein the topically active corticosteriod is administered in combination with prednisone or prednisolone at 2 mg/kg.

9. The method of claim 1 wherein the topically active corticosteroid is formulated for oral administration in the form of a pill, capsule or microsphere.

10. The method of claim 7 wherein the of topically active corticosteroid is formulated such that the pill, microsphere, or capsule dissolves in the stomach, small intestine or colon.

11. The method of claim 1 wherein the topically active corticosteroid is formulated for oral administration in the form of an emulsion.

12. The method of claim 1 wherein administration of the topically active corticosteroid initiates following infusion of the hematopoietic cells.

13. The method of claim 1 wherein administration of the topically active corticosteroid ceases after 80 days following infusion of the hematopoietic cells.

14. The method of claim 1 wherein the patient is the recipient of HLA-mismatched hematopoietic stem cells.

15. The method of claim 1 wherein the patient is the recipient of unrelated donor hematopoietic stem cells, umbilical vein hematopoietic stem cells, or peripheral blood stem cells.

16. The method of claim 1 wherein the topically active corticosteroid is administered in combination with other prophylactic agents.

17. The method of claim 1 wherein the topically active corticosteroid is beclomethasone dipropionate.

18. The method of claim 1 wherein the topically active corticosteroid is alclometasone dipropionate, busedonide, 22S busesonide, 22R budesonide, beclomethasone-17-monopropionate, clobetasol propionate, diflorasone diacetate, flunisolide, flurandrenolide, fluticasone propionate, halobetasol propionate, halcinocide, mometasone furoate, or triamcinalone acetonide.
